(19) United States
(12) Patent Application Publication
(10) Pub. No.: US 2023/0081431 A1
(43) Pub. Date: Mar. 16, 2023

(54) Method For Treating COVID-19 and Related Viral Infections

(71) Applicant: Thomas Walter Comollo, Edison, NJ (US)

(72) Inventor: Thomas Walter Comollo, Edison, NJ (US)

(21) Appl. No.: 17/472,647

(22) Filed: Sep. 12, 2021

(51) Int. Cl. A61K 31/517; A61K 31/502; A61K 31/5377; A61K 31/4245; A61K 31/343; A61K 31/519; A61K 31/538; A61K 31/498; A61K 31/4725; A61K 31/454; A61K 31/496; A61K 31/53; A61K 31/55; A61K 31/4709; A61K 31/5513

(52) U.S. Cl. CPC A61K 31/517; A61K 31/502; A61K 31/5377; A61K 31/4245; A61K 31/343; A61K 31/519; A61K 31/538; A61K 31/498; A61K 31/4725; A61K 31/454; A61K 31/496; A61K 31/53; A61K 31/55; A61K 31/4709; A61K 31/5513

(57) ABSTRACT

A group of known compounds has been found to have polymerase inhibitor qualities and are presumed to be efficacious in the protection of human cells from damage by the SARS-CoV-2 virus, which causes the COVID-19 disease. These compounds, referred to herein as Special Phenyl Compounds, provide such protection by inhibiting replication of the virus, which is done by interfering with its RNA transcription process. These compounds can also be used to treat diseases caused by structurally related viruses such as West Nile virus, Marburg virus, Ebola virus, dengue virus, HIV, hepatitis C virus (HepC), and coronaviruses other than SARS-CoV2. Patients are treated by administering effective doses of one or more of the compounds.

### CROSS-REFERENCE TO RELATED APPLICATIONS

This application claims the benefit of U.S. Provisional Applications Ser. Nos. 63/089,618 and 63/089,629 filed Oct. 9, 2020 and entitled Method For Treating COVID-19 and Related Viral Infections.

### BACKGROUND OF THE INVENTION

This invention relates to a method for treating patients diagnosed with or suffering from infection by the SARS-CoV-2 virus, that is, a COVID-19 infection. The invention is also applicable to treatment of patients infected by structurally similar viruses, such as West Nile virus, Marburg virus, Ebola virus, dengue virus, HIV, hepatitis C virus (HepC), and coronaviruses other than SARS-CoV2.

COVID-19 is a viral respiratory illness, capable of producing a deadly pneumonia that has few, if any, identified pharmacological interventional treatments (other than remdesavir) that specifically target the causative virus's polymerase.

SARS-CoV-2 is the virus that causes the COVID-19 disease. It is a new member of the orthocoronavirinae subfamily, namely genus Sarbecovirus and has a similar structure and replication mechanism as viruses such as West Nile virus, Marburg virus, Ebola virus, dengue virus, HIV, and hepatitis C virus (HepC) which utilize a positive-sense, single strand of RNA to encode their genome.

This genome is replicated within human cells by non-structural protein 12 (nsp12), an RNA-dependent RNA polymerase (RdRp), and is a viral polymerase, similar to reverse transcriptase (RT) in HIV and NS5B in HepC.

The use of RdRp occurs in other positive strand RNA viruses, such as rhinoviruses, that cause common colds, and, not surprisingly, the homologous MERS-CoV and SARS-COV-2 viruses. The existing drug remdesivir targets the SARS-CoV-2 virus's nsp12. Inhibition of RdRp has proven effective in treatment of other viruses, such as HIV and HepC, and has led to compassionate use of remdesivir in the case of SARS-CoV-2.

More targeted treatment is needed for SARS-CoV-2 and the aforementioned structurally related coronaviruses.

### SUMMARY OF THE INVENTION

As herein described, there is provided a method for treating a human or closely biologically related patient diagnosed with or suffering from infection by the SARS-CoV-2 virus or a structurally similar virus, by administering to the patient an effective dose of a substance consisting of a phenyl group containing organic compound selected from the group consisting of
2-(4-oxo-3H-phthalazin-1-yl)-N-(2-phenylquinolin-4-yl)acetamide,
[3-(3,4-dihydro-1H-iso-quinolin-2-ylsulfonyl)phenyl]-[2-(4-fluorophenyl)morpholin-4-yl]methanone, 2-[[2-(3,4-dihydro-2H-1,5-benzodioxepin-7-yl)pyro-lidin-1-yl]methyl]-5-(5-methyl-3-phenyl-1,2-oxazol-4-yl)-1,3,4-oxadiazole, N-dibenzofuran-2-yl-4-(4-oxo-1H-quinazolin-2-yl)but-anamide, N-(9H-fluoren-2-yl)-2-[(4-oxo-1H-quinazolin-2-yl)meth-oxy]acetamide, N-(9H-fluoren-9-yl)-2-[(4-methyl-5-oxo-[1,2,4]tria-zolo[4,3-a]quinazolin-1-yl)sulfanyl]-acetamide, (2S)—N-methyl-4-(3-methyl-4-oxo-2-phenylchromene-8-carbonyl)-2,3-dihydro-1,4-benzoxazine-2-carboxamide, 3-[3-[5-(2-methyl-1,3-thiazol-4-yl)-2,3-dihydroindol-1-yl]-3-oxopropyl]-2H-phthalazine-1,4-dione, (7S)-7-(2,3-dihydro-1,4-benzodioxin-6-yl)-5-[3-(tetrazol-1-yl)phenyl]-2-(trifluoromethyl)-4,7-dihydro-[1,2,4]triazolo[1,5-a]pyrimidine, 3-[[(2S)-2-methyl-2,3-dihydro-indol-1-yl]sulfonyl]-N-(2-methyl-4-oxoquinazolin-3-yl)benzamide, (2R)-4-[2-[5-(4-flu-orophenyl)-4-oxothieno[2,3-d]pyrimidin-3-yl]acetyl]-2,3-dihydro-1,4-benzoxazine-2-carboxamide, (2R)-4-[2-(4-oxo-5-thiophen-2-yl-thieno[2,3-d]pyrimidin-3-yl)acetyl]-2,3-dihydro-1,4-benzoxazine-2-carboxamide, [(1S)-1-(4-oxo-3H-quin-azolin-2-yl)ethyl] 6-cyclo-propyl-3-methyl-[1,2]oxazolo[5,4-b]py-ridine-4-carboxylate, and 4-[2-[4-(5,6,7,8-tetrahydronaphthalen-2-ylsulfonyl)piperazin-1-yl]acetyl]-1,3-dihydroquinoxalin-2-one, (3R)—N-[[4-[(3-fluorophenyl)methoxy]phenyl]methyl]-2-(furan-2-carbonyl)-3,4-dihydro-1H-isoquinoline-3-carboxamide, [(3S)-3-(1,3-benzoxazol-2-yl)piperidin-1-yl]-[3-(2,3-dihydroindo1 ylsulfonyl)phenyl]methanone, [(2S)-1-(2,2-dimethyl-3-oxo-4H-quinoxalin-1-yl)-1-oxopropan-2-yl]9-oxo-2,3-dihydro-1H-pyrrolo[2,1-b]quinazoline-6-carboxylate, 1-[4-[3-[(2R)-2-(2,3-dihydro-1,4-benzodioxin-6-yl)pyrrolidine carbonyl]phenyl]sulfonylpiperazin-1-yl]ethenone, 13-[4-(imidazo[1,2-a]pyridin-2-ylmethoxy)phenyl]-4,8-dioxa-12,14,16,18-tetrazatetracyclo[9.7.0.03,9.012,17]octadeca-1,3(9),10,14,16-pentaen-15-amine, 3-[2-[(2S)-2-(4-methylpiperazine-1-carbonyl)-2,3-dihydro-1,4-benzoxazin-4-yl]-2-oxoethyl]quinazolin-4-one, (3-phenyl-6,7,8,9-tetrahydro-5H-imidazo[1,5-a]azepin-1-yl)-[4-(1H-pyrrolo[2,3-b]pyridin-3-yl)-3,6-dihydro-2H-pyridin-1-yl]methanone, N-(1,3-benzodioxol-5-yl)-1-[2-(4-fluorophenyl)quinoline-4-carbonyl]piperidine-4-carboxamide, [2-(2,2-dimethyl-3-oxo-4H-quinoxalin-1-yl)-2-oxoethyl] 2-(furan-2-yl)quinoline-4-carboxylate, (4S)-4-methyl-5-[2-[(4-oxo-1-phenyl-5H-pyrazolo[3,4-d]pyrimidin-6-yl)sulfanyl]acetyl]-3,4-dihydro-1H-1,5-benzodiazepin-2-one, [2-(2,2-dimethyl-3-oxo-4H-quinoxalin-1-yl)-2-oxoethyl] 3-benzyl-4-oxophthalazine-1-carboxylate.

### IN THE DRAWING

FIGS. 1A to 1K show the structures of the compounds utilized in the treatment method of the present invention.

FIG. 2 shows a bar graph of relative activity of HIV reverse transcriptase in the presence of vehicle (dmso) or 10 μM compound.

FIG. 3 shows the numeric quantitation of the data in FIG. 2.

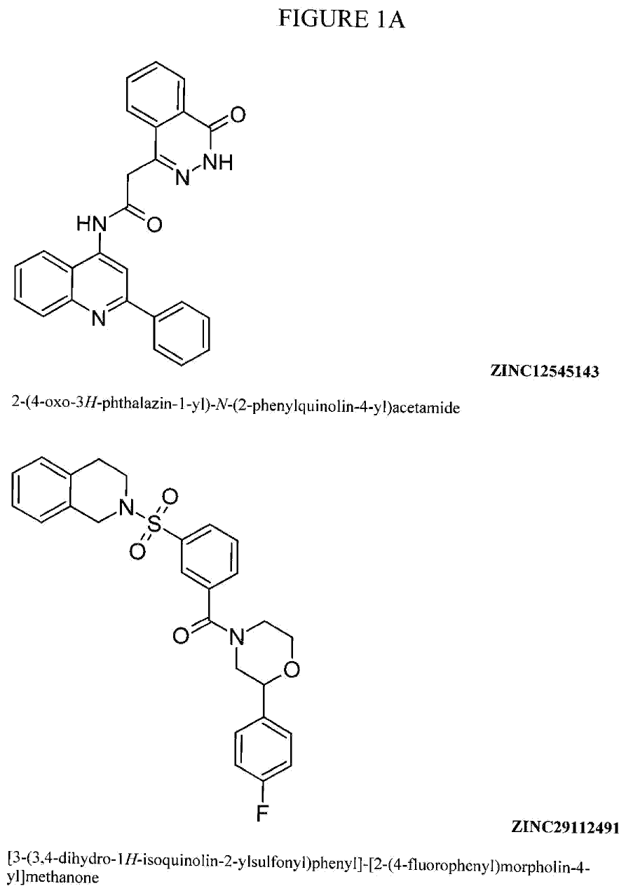

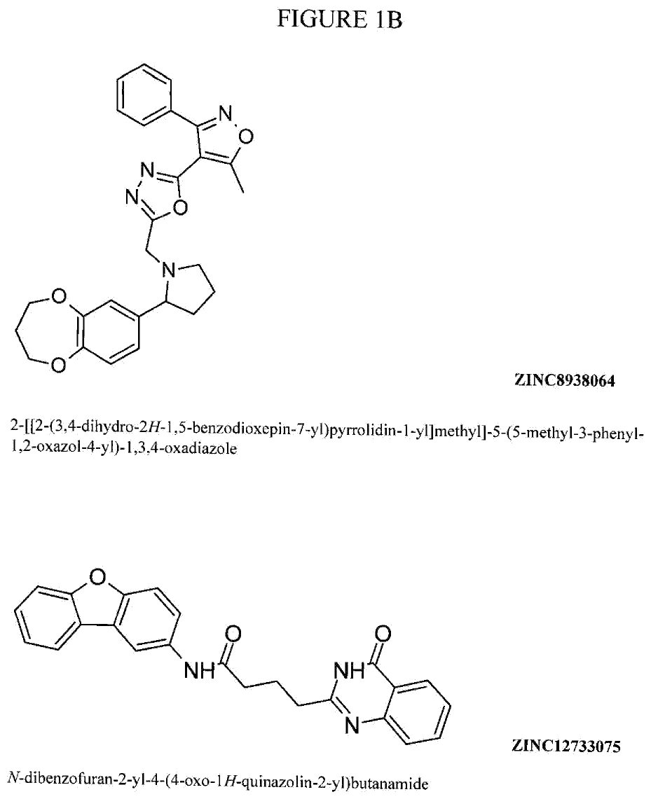

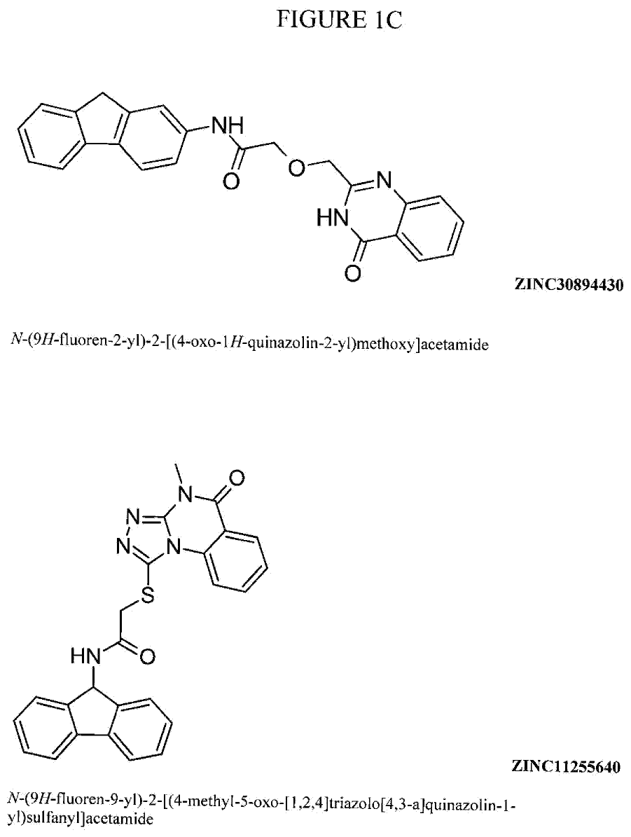

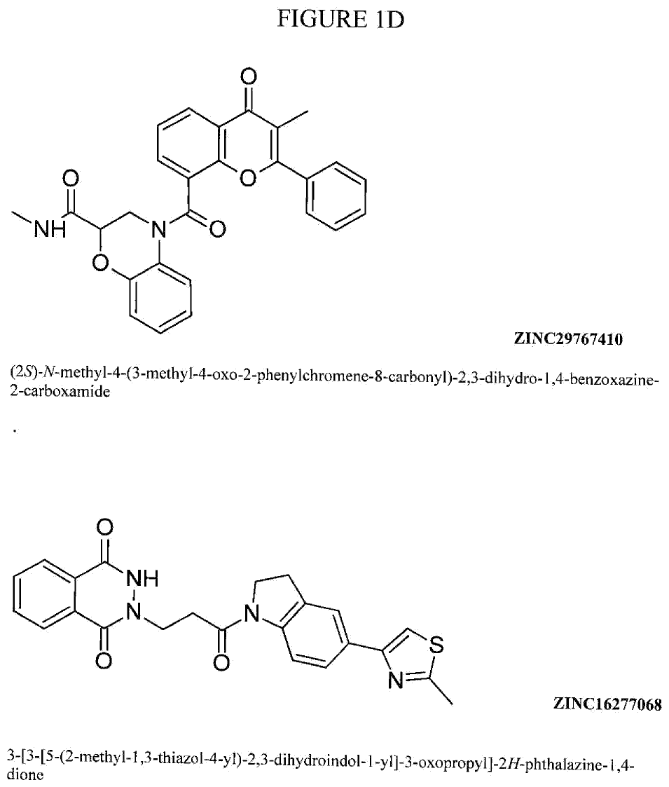

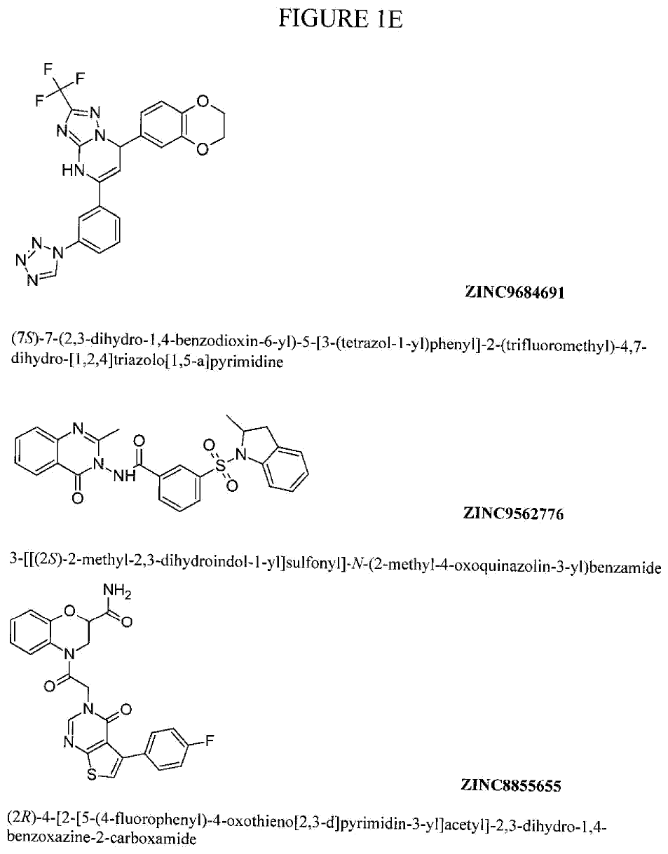

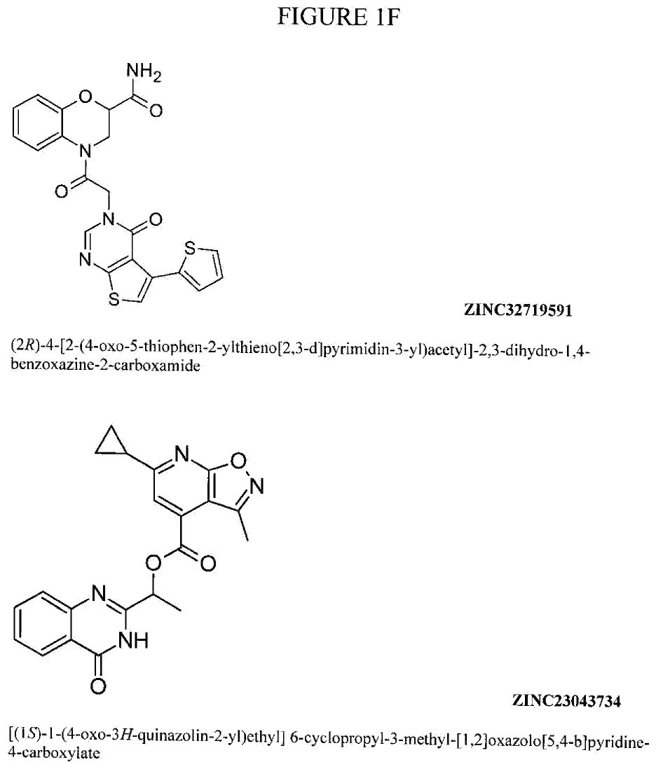

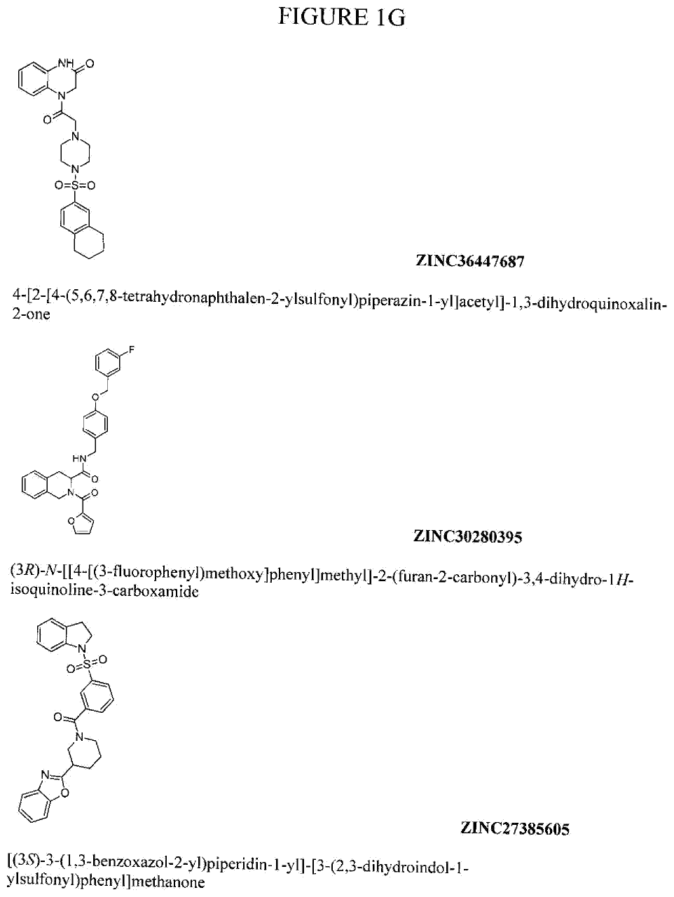

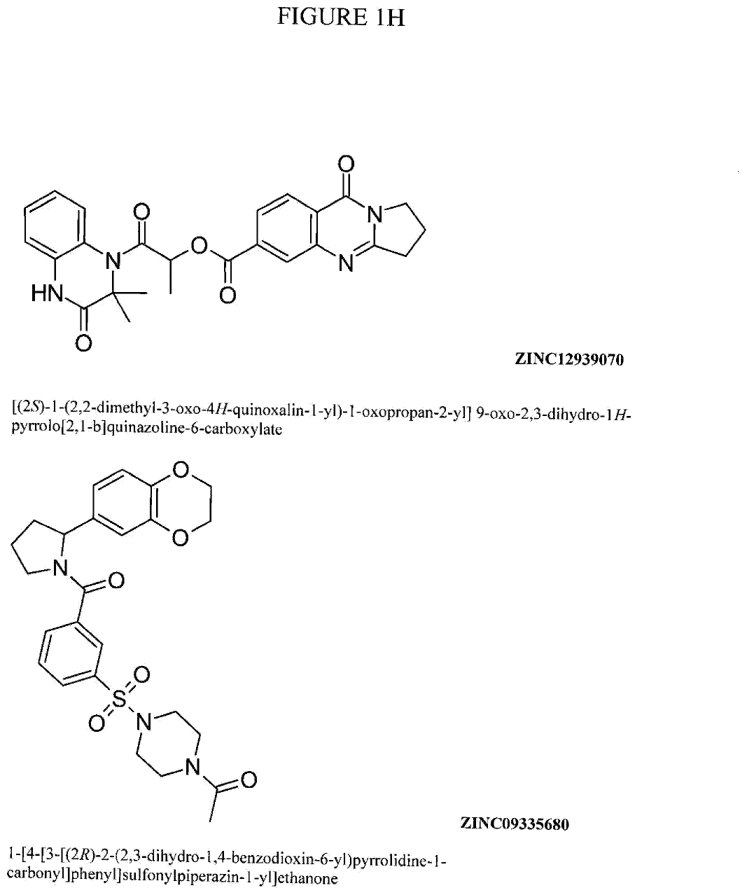

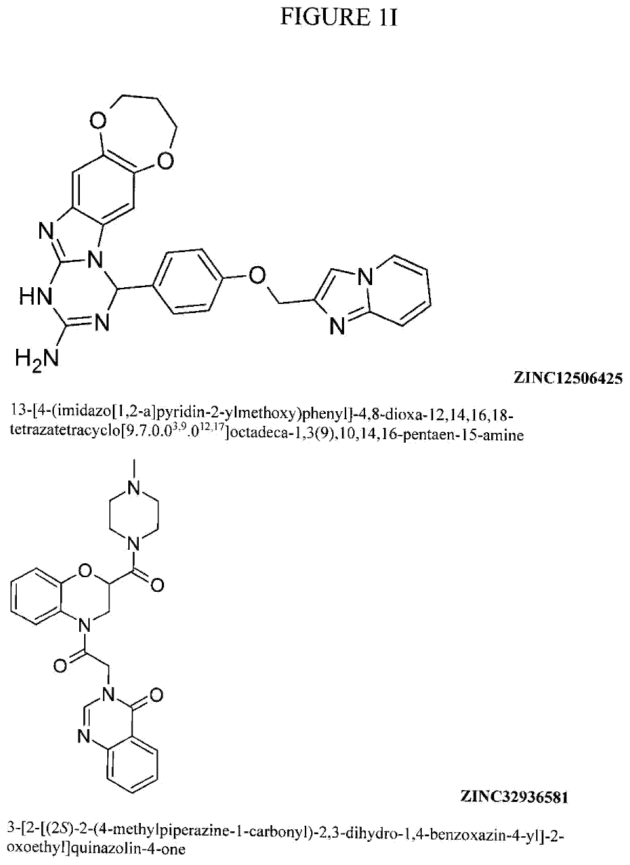

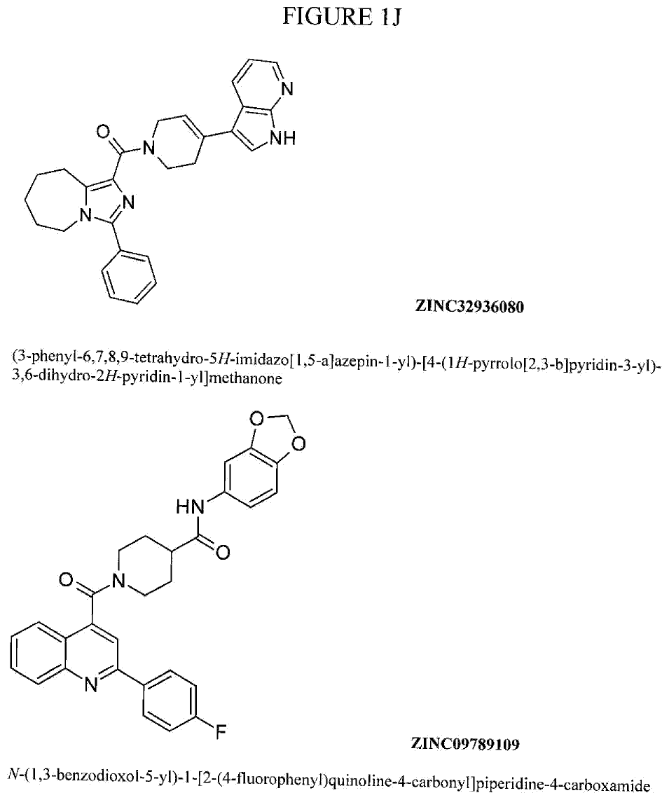

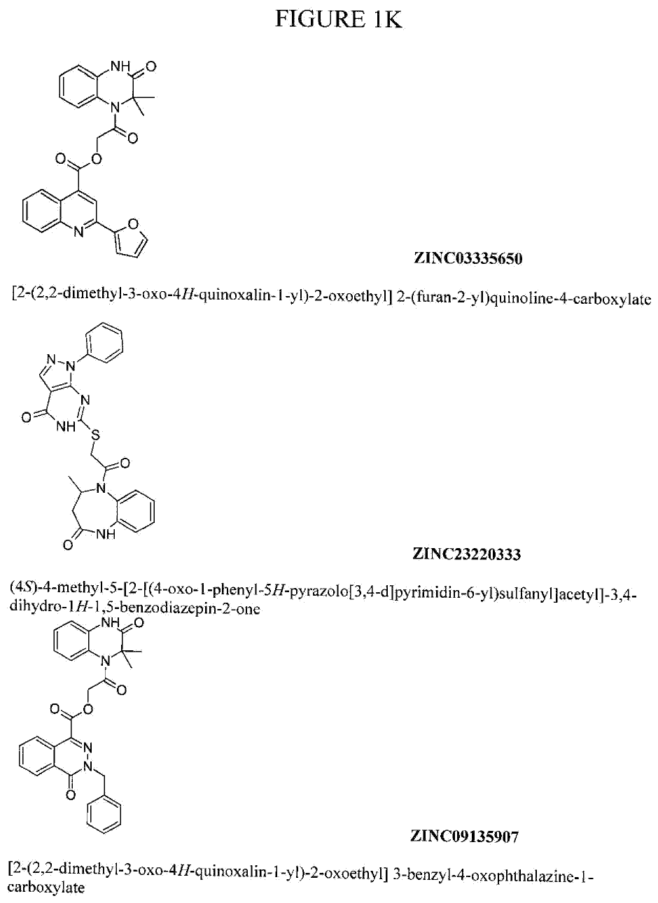

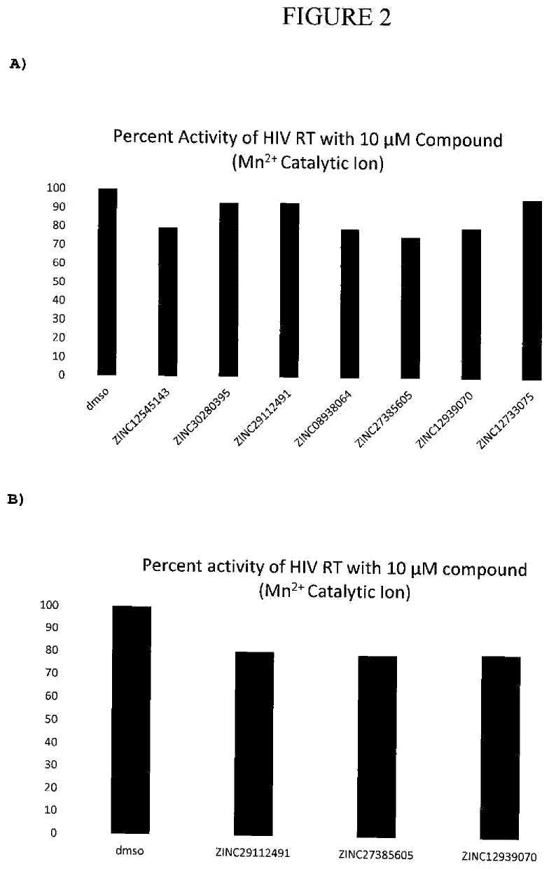

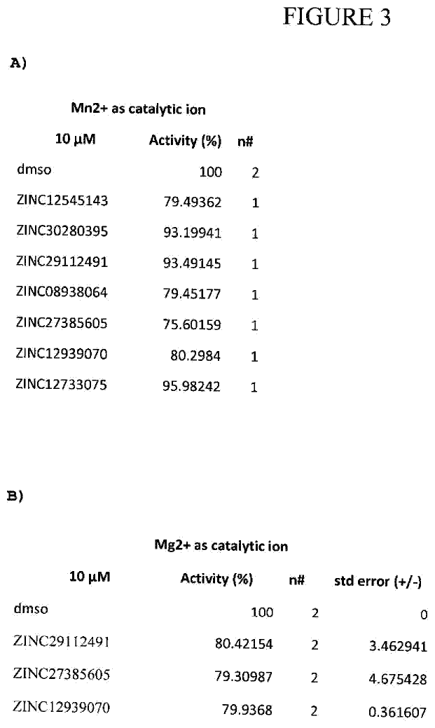

### DETAILED DESCRIPTION

The aforementioned compounds (hereafter the “Special Phenyl Compounds”) have been found to strongly bind the SARS-CoV-2 nsp12 active site in a computational theoretical model. Additionally, compounds from this group have been shown to inhibit the activity of the distantly related polymerase, HIV reverse transcriptase (RT). This anti-viral effect was observed in an in vitro enzymatic assay where each trial consisted of a triplicate of reactions that was averaged and presented as a percentage of vehicle (dmso) treated control from same day/same set of reactions.

The RNA genome of the SARS-CoV-2 and aforementioned related viruses is replicated by an RNA-dependent RNA polymerase (RdRp), or other polymerase, which is non-structural protein 12 (nsp12) in SARS-CoV-2, and is similar to reverse transcriptase in HIV and NS5B in HepC. The Special Phenyl Compounds interfere with this nucleic acid replication by inhibiting the polymerase. These compounds do this somewhat differently than remdesivir, however.

Inhibition of RdRp has proven effective in treatment of other viruses, such as HIV and HepC, and has led to compassionate use of remdesivir, in the case of SARS-CoV-2.

While remdesivir gets incorporated into the nucleotide chain as a nucleotide analog and shuts down the nsp12 enzyme, the Special Phenyl Compounds are believed to bind to areas in and around the catalytic site, inhibiting the binding of the catalytic metal ions and perhaps the binding of template strands and nucleotides as well.

The Special Phenyl Compounds are listed in the ZINC 12 Database (zincl2.docking.dorg) and are available from Enamine, a Ukrainian company having a U.S. location at 1 Distribution Way Monmouth Jct., NJ 08852.

The effective dose can be determined in the usual manner, that is, by clinical testing.

I claim:

1. A method for treating a patient diagnosed with or suffering from infection by the SARS-CoV-2 virus, comprising the steps of:
administering to the patient an effective dose of a substance consisting of a phenyl group containing organic compound selected from the group consisting of 2-(4-oxo-3H-phthalazin-1-yl)-N-(2-phenylquinolin-4-yl)acetamide, [3-(3,4-dihydro-1H-isoquinolin-2-ylsulfonyl)phenyl]-[2-(4-fluorophenyl)morpholin-4-yl]methanone, 2-[[2-(3,4-dihydro-2H-1,5-benzodioxepin-7-yl)pyrolidin-1-yl]methyl]-5-(5-methyl-3-phenyl-1,2-oxazol-4-yl)-1,3,4-oxadiazole, N-dibenzofuran-2-yl-4-(4-oxo-1H-quinazolin-2-yl)but-anamide, N-(9H-fluoren-2-yl)-2-[(4-oxo-1H-quinazolin-2-yl)meth-oxy]acetamide, N-(9H-fluoren-9-yl)-2-[(4-methyl-5-oxo-[1,2,4]triazolo[4,3-a]quinazolin-1-yl)sulfanyl]acetamide, (2S)—N-methyl-4-(3-methyl-4-oxo-2-phenylchromene-8-carbonyl)-2,3-dihydro-1,4-benzoxazine-2-carboxamide, 3-[3-[5-(2-methyl-1,3-thiazol-4-yl)-2,3-dihydroindol-1-yl]-3-oxopropyl]-2H-phthalazine-1,4-dione, (7S)-7-(2,3-dihydro-1,4-benzodioxin-6-yl)-5-[3-(tetrazol-1-yl)phenyl]-2-(trifluoromethyl)-4,7-dihydro-[1,2,4]triazolo[1,5-a]pyrimidine, 3-[[(2S)-2-methyl-2,3-dihydroindol-1-yl]sulfonyl]-N-(2-methyl-4-oxoquinazolin-3-yl)benzamide, (2R)-4-[2-[5-(4-flu-orophenyl)-4-oxothieno[2,3-d]pyrimidin-3-yl]acetyl]-2,3-dihydro-1,4-benzoxazine-2-carboxamide, (2R)-4-[2-(4-oxo-5-thiophen ylthieno[2,3-d]pyrimidin-3-yl)acetyl]-2,3-dihydro-1,4-benzoxa-zine-2-carboxamide, [(1S)-1-(4-oxo-3H-quinazolin-2-yl)ethyl] 6-cyclopropyl-3-methyl-[1,2]oxazolo[5,4-b]pyridine-4-carboxylate, and 4-[2-[4-(5,6,7,8-tetrahydronaphthalen-2-ylsulfonyl)piperazin-1-yl]acetyl]-1,3-dihydroquinoxalin-2-one, (3R)—N-[[4-[(3-fluorophenyl)methoxy]phenyl]methyl]-2-(furan-2-carbonyl)-3,4-dihydro-1H-isoquinoline-3-carboxamide, [(3S)-3-(1,3-benzoxazol-2-yl)piperidin-1-yl]-[3-(2,3-dihydroindo1-1-ylsulfonyl)phenyl]methanone, [(2S)-1-(2,2-dimethyl-3-oxo-4H-quinoxalin-1-yl)-1-oxopropan-2-yl]9-oxo-2,3-dihydro-1H-pyrrolo[2,1-b]quinazoline-6-carboxylate, 1-[4-[3-[(2R)-2-(2,3-dihydro-1,4-benzodioxin-6-yl)pyrrolidine-1-carbonyl]phenyl]sulfonylpiperazin-1-yl]ethenone, 13-[4-(imidazo[1,2-a]pyridin-2-ylmethoxy)phenyl]-4,8-dioxa-12,14,16,18-tetrazatetracyclo[9.7.0.03,9.012,17]octadeca-1,3(9),10,14,16-pentaen-15-amine, 3-[2-[(2S)-2-(4-methylpiperazine-1-carbonyl)-2,3-dihydro-1,4-benzoxazin-4-yl]-2-oxoethyl]quinazolin-4-one, (3-phenyl-6,7,8,9-tetrahydro-5H-imidazo[1,5-a]azepin-1-yl)-[4-(1H-pyrrolo[2,3-b]pyridin-3-yl)-3,6-dihydro-2H-pyridin-1-yl]methanone, N-(1,3-benzodioxol-5-yl)-1-[2-(4-fluorophenyl)quinoline-4-carbonyl]piperidine-4-carboxamide, [2-(2,2-dimethyl-3-oxo-4H-quinoxalin-1-yl) oxoethyl] 2-(furan-2-yl)quinoline-4-carboxylate, (4S)-4-methyl [2-[(4-oxo-1-phenyl-5H-pyrazolo[3,4-d]pyrimidin yl)sulfanyl]acetyl]-3,4-dihydro-1H-1,5-benzodiazepin-2-one, [2-(2,2-dimethyl-3-oxo-4H-quinoxalin-1-yl)-2-oxoethyl] 3-benzyl oxophthalazine-1-carboxylate.

2. The method of claim 1 wherein the patient is human.

3. A method for treating a patient diagnosed with or suffering from infection by the SARS-CoV-2 virus or a structurally similar virus, comprising the steps of:
administering to the patient an effective dose of a substance consisting of a phenyl containing group organic compound selected from the group consisting of 2-(4-oxo-3H-phthalazin-1-y1)—N-(2-phenylquinolin-4-yl)acetamide, [3-(3,4-dihydro-1H-iso-quinolin-2-ylsulfonyl)phenyl]-[2-(4-fluorophenyl)morpholin-4-yl]methanone, 2-112-(3,4-dihydro-2H-1,5-benzodioxepin-7-yl)pyro-lidin-1-yl]methyl1-5-(5-methyl-3-phenyl-1,2-oxazol-4-yl)-1,3,4-oxadiazole, N-dibenzofuran-2-yl-4-(4-oxo-1H-quinazolin-2-yl)but-anamide, N-(9H-fluoren-2-yl)-2-[(4-oxo-1H-quinazolin-2-yl)meth-oxy]acetamide, N-(9H-fluoren-9-yl)-2-[(4-methyl-5-oxo-[1,2,4]triazolo[4,3-a]quinazolin-1-yl)sulfanyl]acetamide, (2S)—N-methyl-4-(3-methyl-4-oxo-2-phenylchromene-8-carbonyl)-2,3-dihydro-1,4-benzoxazine-2-carboxamide, 3-[3-[5-(2-methyl-1,3-thiazol-4-yl)-2,3-dihydroindol-1-yl]-3-oxopropyl]-2H-phthalazine-1,4-dione, (7S)-7-(2,3-dihydro-1,4-benzodioxin-6-yl)-5-[3-(tetrazol yl)phenyl]-2-(trifluoromethyl)-4,7-dihydro-[1,2,4]triazolo[1,5-a]pyrimidine, 3-[[(2S)-2-methyl-2,3-dihydroindol-1-yl]sulfonyl]-N-(2-methyl-4-oxoquinazolin-3-yl)benzamide, (2R)-4-[2-[5-(4-flu-orophenyl)-4-oxothieno[2,3-d]pyrimidin-3-yl]acetyl]-2,3-dihydro-1,4-benzoxazine-2-carboxamide, (2R)-4-[2-(4-oxo-5-thiophen-2-yl-thieno[2,3-d]pyrimidin-3-yl)acetyl]-2,3-dihydro-1,4-benzoxazine-2-carboxamide, [(1S)-1-(4-oxo-3H-quinazolin-2-yl)ethyl] 6-cyclo-propyl-3-methyl-[1,2]oxazolo[5,4-b]pyridine-4-carboxylate, and 4-[2-[4-(5,6,7,8-tetrahydronaphthalen-2-ylsulfonyl)piperazin-1-yl]acetyl]-1,3-dihydroquinoxalin-2-one, (3R)—N-[[4-[(3-fluorophenyl)methoxy]phenyl]methyl]-2-(furan-2-carbonyl)-3,4-dihydro-1H-isoquinoline-3-carboxamide, [(3S)-3-(1,3-benzoxazol-2-yl)piperidin-1-yl]-[3-(2,3-dihydroindo1-1-ylsulfonyl)phenyl]methanone, [(2S)-1-(2,2-dimethyl-3-oxo-4H-quinoxalin-1-yl)-1-oxopropan-2-yl]9-oxo-2,3-dihydro-1H-pyrrolo[2,1-b]quinazoline-6-carboxylate, 1-[4-[3-[(2R)-2-(2,3-dihydro-1,4-benzodioxin-6-yl)pyrrolidine-1-carbonyl]phenyl]sulfonylpiperazin-1-yl]ethenone, 13-[4-(imidazo[1,2-a]pyridin-2-ylmethoxy)phenyl]-4,8-dioxa-12,14,16,18-tetrazatetracyclo[9.7.0.03,9.012,17]octadeca-1,3(9),10,14,16-pentaen-15-amine, 3-[2-[(2S)-2-(4-methylpiperazine-1-carbonyl)-2,3-dihydro-1,4-benzoxazin-4-yl]-2-oxoethyl]quinazolin-4-one, (3-phenyl-6,7,8,9-tetrahydro-5H-imidazo[1,5-a]azepin-1-yl)-[4-(1H-pyrrolo[2,3-b]pyridin-3-yl)-3,6-dihydro-2H-pyridin-1-yl]methanone, N-(1,3-benzodioxol-5-yl)-1-[2-(4-fluorophenyl)quinoline-4-carbonyl]piperidine carboxamide, [2-(2,2-dimethyl-3-oxo-4H-quinoxalin-1-yl) oxoethyl] 2-(furan-2-yl)quinoline-4-carboxylate, (4S)-4-methyl [2-[(4-oxo-1-phenyl-5H-pyrazolo[3,4-d]pyrimidin yl)sulfanyl]acetyl]-3,4-dihydro-1H-1,5-benzodiazepin-2-one, [2-(2,2-dimethyl-3-oxo-4H-quinoxalin-1-yl)-2-oxoethyl] 3-benzyl-4-oxophthalazine-1-carboxylate.

4. The method of claim 3 wherein the patient is human.
